(19) United States
(12) Patent Application Publication
(10) Pub. No.: US 2006/0002983 A1
(43) Pub. Date: Jan. 5, 2006

(54) Muscle building preparation

(75) Inventors: Keitaro Matsumoto, Kagoshima-shi (JP); Tatsuya Doi, Kanzaki-gun (JP); Hiroshi Kitsutaka, Yotsukaido-shi (JP)

Correspondence Address: SUGHRUE MION, PLLC, 2100 PENNSYLVANIA AVENUE, N.W., SUITE 800, WASHINGTON, DC, 20037, US

(73) Assignee: OTSUKA PHARMACEUTICAL CO., LTD., Tokyo 101-8535 (JP)

(21) Appl. No.: 10/534,734

(22) PCT Filed: Nov. 19, 2003
(86) PCT No.: PCT/JP03/14693

§ 371 Date: May 12, 2005

(30) Foreign Application Priority Data
Nov. 20, 2002 (JP) 2002-336202

(51) Int. Cl. A61K 47/00

(52) U.S. Cl. 424/439

(57) ABSTRACT

The present invention provides a muscle-building preparation for use during rehabilitation after injury or post-injury surgery, the preparation containing a composition comprising 42 to 55% by weight of protein, 12 to 20% by weight of fat, and 23 to 42% by weight of carbohydrate, on a dry weight basis of the total weight of the composition; and a food containing the muscle-building preparation.

### TECHNICAL FIELD

The present invention principally relates to a muscle-building preparation effective in increasing muscle mass during rehabilitation after injury or post-injury surgery, and a food comprising the muscle-building preparation.

### BACKGROUND ART

It has become a common practice to perform rehabilitation for restoring rigid joints (arthrogryposis) or weakened muscles (muscular atrophy) after injury or post-injury surgery so as to improve the condition and enhance everyday life of patients after illness or injury.

Rehabilitation is a treatment aiming at recovering from dysfunction caused by surgery, external injuries, and illness such as cerebral hemorrhages and osteoarthritis, and thus needs to be distinguished from training and conditioning in sports. Rehabilitation comprises performing combinations of certain types of exercises, and needs to be distinguished from the treatment mainly consisting of recuperation for recovery from illnesses or surgery.

From such a viewpoint, special nutritional management is required during rehabilitation to promote recovery from injuries and illnesses.

Heretofore, there are known nutritional supplementary foods such as a food composition containing protein, fat and carbohydrate (Japanese Examined Patent Publication No. 1995-102112).

However, compositions particularly suitable for promoting muscle building and recovery in rehabilitation after injury or post-injury surgery have yet to be achieved.

### DISCLOSURE OF THE INVENTION

The object of the invention is to provide a preparation and a method which are effective for promoting injury recovery and improving the condition of patients, particularly for increasing muscle mass, in rehabilitation after injury or post-injury surgery.

The present inventors carried out intensive research, focusing attention on the following observation: in order to achieve injury recovery or condition improvement of patients in rehabilitation after injury or post-injury surgery, pharmaceutical preparation or foods having more appropriate nutritional compositions accounting of the condition of patients and physical exercise are required, instead of nutritional compositions as generally ingested during sports or resting for recovery from illness.

As a result, the inventors found that a composition having certain specific constitution is effective in enhancing rehabilitation effects after injury or post-injury surgery and promoting muscle building. The inventors conducted further research based on this finding and thereby accomplished the invention.

The present invention relates to the following muscle-building preparations, foods, and methods of building muscles.

Item 1. A muscle-building preparation for use during rehabilitation after injury or post-injury surgery, the preparation containing a composition comprising 42 to 55% by weight of protein, 12 to 20% by weight of fat, and 23 to 42% by weight of carbohydrate, on a dry weight basis of the total weight of the composition.

Item 2. The muscle-building preparation according to item 1 wherein the injury is a fracture, joint injury, pulled muscle, or sprain.

Item 3. The muscle-building preparation according to item 1 wherein the injury is a ligament injury.

Item 4. A food comprising the muscle-building preparation of item 1.

Item 5. A food indicated as a food for muscle building in rehabilitation after injury or post-injury surgery, the food containing a composition comprising 42 to 55% by weight of protein, 12 to 20% by weight of fat, and 23 to 42% by weight of carbohydrate, on a dry weight basis of the total weight of the composition.

Item 6. A method of building muscles during rehabilitation after injury or post-injury surgery, comprising the step of ingesting a muscle-building preparation for use during rehabilitation after injury or post-injury surgery within 1 hour after physical exercise or a food comprising the preparation, the preparation comprising a composition comprising 42 to 55% by weight of protein, 12 to 20% by weight of fat, and 23 to 42% by weight of carbohydrate, on a dry weight basis of the total weight of the composition.

Item 7. The method according to item 6 wherein the injury is a fracture, joint injury, pulled muscle, or sprain.

Item 8. The method according to item 6 wherein the injury is a ligament injury.

The present invention is described below in detail.

Muscle-Building Preparation

The muscle-building preparation of the invention contains a composition as an active agent, the composition having the following specific constitution: 42 to 55% by weight of protein, 12 to 20% by weight of fat, and 23 to 42% by weight of carbohydrate on a dry weight basis of the total weight of the composition.

Ingestion of the muscle-building preparation of the invention during rehabilitation after injury or post-injury surgery can effectively increase muscle mass without placing an excessive burden on the body.

In this specification, “%” indicates “weight %”, unless otherwise specified.

Protein

The composition of the invention contains protein as an essential ingredient. Protein is one of the three major nutrients, along with carbohydrate and fat, and serves as a body constituent material. Protein also serves as an energy source, along with carbohydrate and fat.

Examples of protein source usable in the invention include whey protein concentrates (WPCs), whey protein isolates (WPIs), desalted whey, casein and salts thereof, gelatin and salts thereof, water-soluble gelatins (enzymatic hydrolysable gelatins, etc.), whole milk powders, skim milk powders, soybean protein, wheat protein, corn gluten and the like.

Such proteins can be used singly or in combination of two or more.

WPC and WPI are whey products obtained by subjecting liquid whey, which is a by-product produced during the production of milk products such as cheese and casein, to operations such as filtration, ion exchange, crystallization, precipitation and/or reverse osmosis. Although there are slight differences depending on the manufacturer, their various physical properties, including their protein composition, are approximately as shown in Table 1 (see New Food Industry, 25 (3), 33 (1983), etc.).

TABLE 1

| | WPC- | WPC- | WPC- | WPC- | WPC- | |
|---|---|---|---|---|---|---|
| | 34 | 50 | 60 | 75 | 80 | WPI |
| Protein | 34-36 | 50-52 | 60-62 | 75-78 | 80-82 | 90-92 |
| α-lactoglobulin | 6.5 | 9.5 | 11 | 14 | 15 | 21 |
| β-lactoglobulin | 16 | 24 | 29 | 36 | 38 | 47 |
| Serum albumin | 1.7 | 2.5 | 3.0 | 3.8 | 4.0 | 1.5 |
| Immuno- | 2.7 | 4.0 | 4.8 | 6.0 | 6.4 | 2.4 |
| globulin | | | | | | |
| Lactose | 48-52 | 33-37 | 25-30 | 10-15 | 4-8 | 0.5-1 |
| Fat | 3-4.5 | 5-6 | 1-7 | 4-9 | 4-8 | 0.5-1 |
| Ash | 6.5-8.0 | 4.5-5.5 | 4-6 | 4-6 | 3-4 | 2-3 |
| Water | 3.0-4.5 | 3.5-4.5 | 3-5 | 3-5 | 3.5-4.5 | 4.5 |
| PH | 6-6.7 | 6-6.7 | 6-6.7 | 6-6.7 | 6-6.7 | 6-6.7 |

Desalted whey is obtained by pasteurizing whey at a low temperature and removing therefrom minerals by precipitation, filtration, dialysis and/or other separation techniques. Desalted whey usually contains about 79% carbohydrate, about 2% fat, about 13% protein and less than about 7% ash.

When WPC, WPI or desalted whey is used, the proportion of protein in the composition of the invention is indicated by the amount of protein in the WPC, WPI or desalted whey.

Fat

Fat is a nutrient used as an energy source for physical exercise. Fat is used in a different manner from carbohydrate and serves as an important energy source during long-term physical exercise, in particular. Moreover, fat is used as a source for hormone synthesis and has a role in transporting fat-soluble vitamins.

Examples of fats usable in the invention include soybean oil, cottonseed oil, safflower oil, corn oil, rice oil, coconut oil, basil oil, sesame oil, linseed oil, and like vegetable oils; sardine oil, cod liver oil, and like fish oils, toad oil and the like. Medium-chain fatty acid triglycerides (MCTs), which are triglycerides of fatty acids usually having 8 to 10 carbon atoms, are also usable. MCTs are characterized by easy absorptive property, preferential oxidative property and lower potential for fat deposition.

Such fats can be used singly or in combination of two or more members of the same or different groups.

Carbohydrate

Carbohydrate is stored as glycogen in the liver and muscles, and consumed as an energy source during exercise, etc.

Specific examples of carbohydrates include monosaccharides such as glucose and fructose; disaccharides such as maltose and sucrose; sugar alcohols such as xylitol, sorbitol, glycerol and erythritol; polysaccharides such as dextrin and cyclodextrin; and oligosaccharides such as fructooligosaccharide and galactooligosaccharide.

Such carbohydrates can be used singly or in combination of two or more. When two or more carbohydrates are used in combination, commercially available carbohydrate mixtures, such as isomerized sugar and purified sucrose, are also usable.

Proportions of the Three Components

The proportions of the above three components in the composition of the muscle-building preparation of the invention are preferably selected from the ranges shown in Table 2.

TABLE 2

| | Acceptable | Suitable |
|---|---|---|
| | proportions | proportions |
| Component | (%) | (%) |
| Protein | 42-55 | 42-52 |
| Fat | 12-20 | 14-18 |
| Carbohydrate | 23-42 | 30-40 |

Compositions comprising components in the above proportions are particularly effective for increasing muscles mass during rehabilitation after injury or post-injury surgery.

For example, when the proportion of protein is higher than that shown in Table 2 and the proportions of fat and carbohydrate relative to protein are lower than those shown in Table 2, energy metabolism efficiency is adversely affected, and the burden on the liver and kidneys caused by protein decomposition increases, thus causing an excessive burden on the body and impairing recovery from injury.

In contrast, when the proportion of protein is lower than that shown in Table 2 and the proportions of fat and carbohydrate relative to protein are higher than those shown in Table 2, the effects of a recovery of weakened muscles and a rehabilitation are impaired.

When the proportions of fat and carbohydrate are outside the ranges shown in Table 2, efficient energy metabolism suited to mid- and long-term exercises such as rehabilitation is not achieved, thus causing problems such as delayed recovery from injury or illness and increased burden on the body.

Other Components

The composition of the invention may further contain appropriate additives, if desired. Examples of usable additives include emulsifying agents, gelling agents and/or thickening agents.

The composition of the invention preferably comprises an emulsifying agent. Since lipids are soluble in oil but sparingly soluble in water, fat is used in the form of an oil-in-water emulsion in the invention. Therefore, in preparing the composition of the invention comprising fat as an essential component, an emulsifying agents is preferably used to emulsify the fat.

Examples of emulsifying agents are glycerol fatty acid esters. Examples of useful glycerol fatty acid esters include various esters known to be usable as emulsifying agents in this type of food industry. For example, any of highly purified monoglycerides, reactive monoglycerides, fatty acid monoesters of highly purified diglycerol and polyglycerol esters can be used. Specific examples include commercially available “Sunsoft” (trademark, manufactured by TAIYO KAGAKU CO., LTD.), “Emulsy” (trademark, manufactured by RIKEN VITAMIN CO., LTD.) and “Ryoto” (trademark, manufactured by MITSUBISHI-KAGAKU FOOD CORPORATION).

It is also possible to use emulsifying agents which are used in this type of food industry other than glycerol fatty acid esters. Examples thereof include phospholipids such as egg yolk lecithin, hydrogenated egg yolk lecithin, soybean lecithin, and hydrogenated soybean lecithin; synthetic surfactants such as polyoxyethylene monooleates (e.g., commercially available product “Tween 80” (manufactured by AMR)), sucrose fatty acid esters, sorbitan fatty acid esters, propylene glycol fatty acid esters, and the like.

Such emulsifying agents can be used singly or in combination of two or more. The combined use of two or more emulsifying agents is usually preferable.

The proportion of emulsifying agents in the composition of the invention is about 0 to about 0.5 wt. %, and preferably about 0 to about 0.3 wt. %. When the composition of the invention is produced by preparing in advance an emulsion or dispersion mixture of protein, citric acid and other acidic components, the proportion of emulsifying agents in the emulsion or dispersion is preferably in the range of about 1 to about 5%, and more preferably about 3 to about 5%.

Further adding a gelling agent and/or a thickening agent to the composition is preferable in that excellent body function improvement and muscle-building effects can be achieved.

Examples of gelling agents include agar, gellan gum, carrageenan, pectin, gelatin and the like.

Examples of thickening agents include furcellaran, locust bean gum, guar gum, gum Arabic, xanthan gum and the like.

Such gelling agents and thickening agents can be used singly or in combination of two or more. A combination of a gelling agent and a thickening agent, such as agar and a thickening agent, is particularly preferable. Such gelling agents and/or thickening agents exhibit an appropriate gelling ability and gel stabilizing ability and also produce effects such as improved water releasing capability and excellent texture.

Preferably, gelling agent and thickening agent are usually incorporated in a proportion range from about 0.05 to 3 wt. % each in the composition of the invention.

The composition of the invention may further contain other additives, for example, sweeteners such as natural sweeteners (other than carbohydrates) and artificial sweeteners, vitamins and minerals (electrolytes and trace elements), flavoring agents such as natural flavors and synthetic flavors, coloring agents, flavors (chocolate, etc.), food preservatives, natural fruit juices, natural fruit fleshes, and the like. Among these additives, addition of vitamins and/or minerals is particularly preferable in terms of excellent effects of muscle building and amelioration of pathological conditions.

Examples of natural sweeteners (other than carbohydrates) include thaumatin, stevia extracts (rebaudioside A, etc.), glycyrrhizin, and the like. Examples of artificial sweeteners include saccharin, aspartame, and the like.

Examples of vitamins include water-soluble and fat-soluble vitamins, such as vitamin A (retinols), vitamin B₁ (thiamine), vitamin B₂ (riboflavin), vitamin B₆ (pyridoxine), vitamin B₁₂ (cyanocobalamin), vitamin E (tocopherol), niacin, bisbentiamine, nicotinamide, calcium pantothenate, folic acid, biotin, choline bitartrate, and the like.

Examples of particularly preferable vitamins include multivitamins having the following composition ranges:

| | | |
|---|---|---|
| Vitamin A | 10-2000 | IU |
| Vitamin B₁ | 0.01-3.0 | mg |
| Vitamin B₂ | 0.01-3.1 | mg |
| Vitamin B₆ | 0.01-3.2 | mg |
| Vitamin B₁₂ | 0.1-30 | μg |
| Vitamin C | 1-50 | mg |
| Vitamin D | 10-100 | IU |
| Vitamin E | 1-100 | IU |
| Niacin | 0.1-30 | mg |
| Calcium | 0.1-31 | mg |
| pantothenate | | |
| Folic acid | 0.01-3.0 | mg |

Examples of minerals (electrolytes and trace elements) include those generally used in the food industry such as sodium chloride, sodium acetate, magnesium sulfate, magnesium chloride, dipotassium phosphate, monosodium phosphate, ferric citrate, ferrous pyrophosphate, ferric pyrophosphate, sodium iron succinatocitrate, manganese sulfate, cupric sulfate, zinc sulfate, sodium iodide, potassium sorbate, zinc, manganese, copper, iodine, cobalt, and the like.

Examples of flavoring agents, such as natural and synthetic flavors include apple flavors, orange flavors, grapefruit flavors, lemon flavors, and the like.

Examples of coloring agents include Red No. 2, Red No. 3, Green No. 3, Blue No. 1, Blue No. 2, Yellow No. 4, Yellow No. 5, red cabbage pigment, orange pigment, gardenia pigment, chlorophyll, Perilla color, tomato pigment, safflower pigment and the like.

Examples of flavors include chocolate.

Examples of preservatives include butyl hydroxyanisole (BHA), dibutylhydroxytoluene (BHT), sodium nitrate, sodium nitrite, disodium ethylenediaminetetra-acetate (EDTA), tert-butylhydroquinone (TBHQ), benzoic acid, Japanese styrax benzoin extract, Rumput Roman extract, hinokitiol extract, pectin digests, Magnolia obovata extract, Forsythia extract and the like.

Examples of natural fruit juices and natural fruit fleshes include those of apples, green apples, oranges, mandarins, grapefruits, peaches, strawberries, muscats, grapes, pineapples, lemons, pears, lychees, blueberries, mangos, bananas, and like fruits.

Such additives can be used singly or in combination of two or more. The proportion of each additive can be suitably determined as desired.

Production Process

The muscle-building preparation of the invention can be obtained by mixing the above components and using the resulting composition as is or by combining the resulting composition with carriers. The method of preparing the composition is not particularly limited, and known methods can be appropriately used. Neither is the form of the composition particularly limited. For example, a viscous composition can be prepared.

For example, the composition used in the invention can be prepared by adding protein, carbohydrate and like auxiliary emulsifiers, and if necessary, lecithin, sugar esters and like conventional emulsifying agents, to the oil-soluble components (e.g., fats, oils, and other oil-soluble materials), and emulsifying the mixture by a conventional method. The examples of the emulsification method include mechanical emulsification. The composition thus obtained is placed in a suitable container and retort-sterilized (120° C., 20 minutes, for example) to provide an end product with a good shelf-life. This product can be used directly or after appropriate dilution.

The composition prepared in the above manner is digested and absorbed in the intestinal tract at suitable rates, and has a low osmotic pressure during the digestion and absorption, thus the person taking the composition almost never has a risk of diarrhea, and the composition can constantly stabilize and sufficiently provide its intrinsic nutritional status improving effects.

The intake volume of the composition is preferably selected on a dry weight basis from the range of about 10-30 g/dose, and more preferably about 15-25 g/dose, and in total volume from the range of about 50-300 cc, and more preferably about 150-250 cc.

The composition used in the invention may also be prepared as a gel composition.

The gel composition can be prepared by mixing and emulsifying the specified amounts of components mentioned above, a gelling agent and/or a thickening agent, and a certain amount of water with heating, followed by cooling. Such emulsification can be conducted by adding all the components to water at once and then carrying out lightly a mechanical operation such as stirring. Alternatively, it can be conducted by preliminarily preparing an aqueous solution of water-soluble components, adding thereto oil-soluble components and an emulsifying agent or a mixture thereof, and subjecting them to a similar mechanical operation such as stirring. The latter is preferable to obtain a more uniformly emulsified mixture. The above mixing operation (emulsification operation) may be performed at room temperature, but is preferably performed while heating. The emulsifying operation can be conducted in a conventional manner with a suitable homogenizer such as a homomixer, high-pressure homogenizer or the like. The emulsifying operation can be conducted by complete passage process or circulation process.

Foods

The invention can be provided not only as the above muscle-building preparation but can also be provided in the form of a food comprising the muscle-building preparation.

The form of food is not particularly limited. For example, it can be a viscous food composition or gel-form food composition. Such viscous food compositions and gelatinous food compositions may be placed in a suitable container, sterilized and then cooled. Examples of containers suitable for viscous food compositions include tubes. Examples of containers suitable for gel compositions include pouch containers such as a Spouch.

The proportion of the muscle-building preparation in such foods can be suitably selected according to the use and purpose.

In addition to the above components, the food of the invention may, if desired, additionally contain appropriate food materials and components typically added to foods.

As with the muscle-building preparation, intake of the food during rehabilitation after injury or post-injury surgery can effectively increase muscle mass without placing an excessive burden on the body, and is thus effectively usable as a muscle-building food during rehabilitation after injury or post-injury surgery.

The food of the invention contains the composition described above as an active ingredient of the muscle-building preparation. The food of the invention can be used as a food indicated to be used for muscle building in rehabilitation after injury or post-injury surgery, the food containing a composition comprising 42 to 55% by weight of protein, 12 to 20% by weight of fat, and 23 to 42% by weight of carbohydrate, on a dry weight basis of the total weight of the composition.

Injuries

Intake of the muscle-building preparation or food of the invention is suited to rehabilitating injuries, for example, fractures, joint injuries, pulled muscles, and sprains. Examples of joint injuries include knee joint injuries and ligament injuries. More specifically, collateral ligament ruptures, anterior cruciate ligament ruptures, porterior cruciate ligament ruptures and like ligament ruptures and medial meniscus injuries can be mentioned as examples. The invention is particularly suitable for ligament ruptures such as anterior cruciate ligament ruptures.

The intake of the muscle-building preparation or food containing the muscle-building preparation of the invention during rehabilitation after such an injury or post-injury surgery can effectively increase muscle mass without placing an excessive burden on the body.

The route of intake is preferably oral ingestion.

With regard to the timing of intake, it should be taken within several hours before or after physical exercise, preferably within 1 hour, particularly preferably within several minutes immediately after rehabilitation exercise. Intake in the intervals between rehabilitation exercises is also preferable.

By ingesting the muscle-building preparation or food containing the muscle-building preparation during rehabilitation after injury or post-injury surgery in such a manner, muscle mass can be effectively increased without placing an excessive burden on the body. In particular, the invention can remarkably increase muscle mass at sites proximal to the injury site, more specifically, muscles within the range of 20 cm from injury, and more suitably, within 10 cm.

### BRIEF DESCRIPTION OF THE DRAWINGS

FIG. 1 is a diagram showing the results of measuring cross-sectional area of muscle changes achieved by the intake of a product of the invention and comparative products during rehabilitation after injury, which are expressed as a rate of increase in thigh muscle cross-sectional area (%). P-value indicates the statistical significance level.

### BEST MODE FOR CARRYING OUT THE INVENTION

Examples and Comparative Examples are provided below to illustrate the present invention in further detail. In the examples, parts and percentages are all by weight unless otherwise specified.

### EXAMPLES AND COMPARATIVE EXAMPLES

The compositions of Example 1 and Comparative Examples 1 and 2 shown in Table 3 below were diluted with water to a volume of 80 ml, and then placed in tube-type containers and sterilized to provide pharmaceutical preparations.

With respect to Example 2, the composition of Example 2 shown in Table 3 was diluted with water to a volume of 200 ml, and then placed in a Spouch (manufactured by Dai Nippon Printing Co., Ltd.) and sterilized by heating at 80° C. for 10 minutes, followed by cooling to provide a pouched gel composition.

In Table 3, the components of the compositions are expressed on a dry weight basis. With respect of IUs in Table 3, the following conversion coefficient can be used: 1 IU of vitamin A=0.3 μg, 1 IU of vitamin D=0.025 μg, and 1 IU of vitamin E=1 mg.

TABLE 3

| | Example 1 | Example 2 | Comp. Ex. 1 | Comp. Ex. 2 |
|---|---|---|---|---|
| Protein (g) | 10 | 9 | 0 | 0 |
| Carbohydrate (g) | 7 | 8 | 17 | 1.4 |
| Fat (g) | 3.3 | 3.3 | 3.3 | 1 |
| Emulsifying | 0 | 0.02 | 0 | 0 |
| agent (g) | | | | |
| Gelling agent (g) | 0 | 0.3 | 0 | 0 |
| Thickening | 0 | 0.1 | 0 | 0 |
| agent (g) | | | | |
| Minerals | | | | |
| Sodium (Na, mg) | 220 | 220 | 220 | — |
| Calcium (Ca, mg) | 120 | 120 | 120 | — |
| Iron (Fe, mg) | 0.8 | 0.8 | 0.8 | — |
| Potassium (K, mg) | 150 | 150 | 150 | — |
| Phosphorus (P, mg) | 160 | 160 | 160 | — |
| Vitamins | | | | |
| Vitamin A (IU) | 670 | 670 | 670 | — |
| Vitamin B1 (mg) | 0.3 | 0.3 | 0.3 | — |
| Vitamin B2 (mg) | 0.5 | 0.5 | 0.5 | — |
| Vitamin B6 (mg) | 0.5 | 0.5 | 0.5 | — |
| Vitamin B12 (μg) | 0.7 | 0.7 | 0.7 | — |
| Niacin (mg) | 5.7 | 5.7 | 5.7 | — |
| Pantothenic | 1.8 | 1.8 | 1.8 | — |
| acid (mg) | | | | |
| Folic acid (mg) | 0.6 | 0.6 | 0.6 | — |
| Vitamin C (mg) | 17 | 17 | 17 | — |
| Vitamin D (IU) | 65 | 65 | 65 | — |
| Vitamin E (IU) | 2.7 | 2.7 | 2.7 | — |
| Calories (kcal) | 100 | 100 | 100 | 15 |

Evaluation Method

To establish the effects of the invention, the effects of intaking preparations of Example 1 and Comparative Examples 1 and 2 (test foods) were compared and studied using sports players with anterior cruciate ligament (ACL) injuries (17 players, age: 25±5 years old (mean±standard deviation) as subjects.

The subjects got resistance training by leg stretching and bending exercises (three times per week) for 12 weeks. Immediately after each bout of training, the subjects ingested one serving of test foods.

The subjects were divided at random into the following three groups: group A (given the test food of Example 1) group B (given the test food of Comparative Example 1), and group C (given the test food of Comparative Example 2).

To diminish the influence of daily diet on the effects of test food ingestion after exercise, the intake of foods other than the test foods and water was prohibited during the period from 2 hours before to 2 hours after training. The thigh muscle cross-sectional area of the subjects was measured as an index of muscle mass (at the proximal site: 10 cm from injury site; intermediate site: 20 cm from injury site; distal site: 30 cm from injury site) before and after 12-week training period.

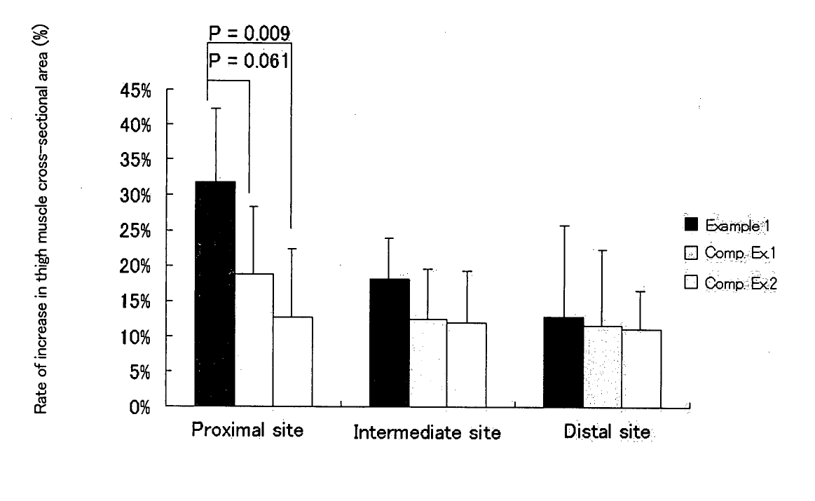

The muscle cross-sectional area was determined by subjecting images obtained by the MRI (magnetic resonance imaging) method to computer processing to compute the muscle area. FIG. 1 shows the results.

The rate of increase in thigh muscle cross-sectional area (%) of group A at the proximal site, 10 cm from the injury site (ACL), was significantly higher than that of group C, and tended to be higher than that of group B (group A: 31.9±10.5%, group B: 18.2±5.8%, group C: 12.9±13.0%; group A vs B=0.061, Group A vs Group C=0.009).

Statistical comparisons among the groups were performed by Fisher's PLSD tests.

Similarly to the proximal site, the rate of increase in muscle cross-sectional area (%) at the intermediate site, 20 cm from the injury site (ACL), were such that groups A, B and C were from high to low in that order (group A: 18.8±9.5%, group B: 12.5±7.1%, group C: 11.6±10.7%).

Similar results were also obtained at the distal site, 30 cm from injury site (ACL), (group A: 12.7±9.6%, group B: 12.0±7.3%, group C: 11.1±5.6%).

### INDUSTRIAL APPLICABILITY

Muscle building and amelioration of pathological conditions during rehabilitation after injury or post-injury surgery can be efficiently achieved by the intake of the muscle-building preparation of the invention. The present invention is particularly suitable for use in rehabilitation of joint injuries including ligament injuries such as ligament ruptures, fractures, pulled muscles, sprains, etc.

Intake of the muscle-building preparation or a food comprising the muscle-building preparation during rehabilitation after injury or post-injury surgery can efficiently increase muscle mass without placing an excessive burden on the body after injury or post-injury surgery. In particular, the preparation and food of the invention can remarkably build muscles at sites proximal to the injury site.

The muscle-building preparation or food of the invention having such properties can be effectively utilized in enhancing or improving rehabilitative effects after injury or post-injury surgery.

1. A muscle-building preparation for use during rehabilitation after injury or post-injury surgery, the preparation comprising a composition comprising 42 to 55% by weight of protein, 12 to 20% by weight of fat, and 23 to 42% by weight of carbohydrate, on a dry weight basis of the total weight of the composition.

2. The muscle-building preparation according to claim 1 wherein the injury is a fracture, joint injury, pulled muscle, or sprain.

3. The muscle-building preparation according to claim 1 wherein the injury is a ligament injury.

4. A food comprising the muscle-building preparation of claim 1.

5. A food indicated as a food for muscle building during rehabilitation after injury or post-injury surgery, the food containing a composition comprising 42 to 55% by weight of protein, 12 to 20% by weight of fat, and 23 to 42% by weight of carbohydrate, on a dry weight basis of the total weight of the composition.

6. A method of building muscles during rehabilitation after injury or post-injury surgery, comprising the step of ingesting a muscle-building preparation for use during rehabilitation after injury or post-injury surgery, or a food comprising the preparation, within 1 hour after physical exercise, the preparation comprising a composition comprising 42 to 55% by weight of protein, 12 to 20% by weight of fat, and 23 to 42% by weight of carbohydrate, on a dry weight basis of the total weight of the composition.

7. The method according to claim 6 wherein the injury is a fracture, joint injury, pulled muscle, or sprain.

8. The method according to claim 6 wherein the injury is a ligament injury.
